SEQUENCE LISTING (40 sequences)

SEQ ID NO: 1 - DNA, 96 bp, Unknown
  oligonucleotide
aattcaaggagttaattatgaaaaaaaccgcgattgcgattgcggtggcgctggcgggctttgcgaccgtggcccaggcggcctctagaatctgcg

SEQ ID NO: 2 - DNA, 108 bp, Unknown
  oligonucleotide
gccgcactagtgcggccgcagattctagaggccgcctgggccacggtcgcaaagcccgccagcgccaccgcaatcgcaatcgcggtttttttcataattaactccttg

SEQ ID NO: 3 - DNA, 36 bp, Unknown
  oligonucleotide
ggagtctagataactgtggctgcaccatctgtcttc

SEQ ID NO: 4 - DNA, 37 bp, Unknown
  oligonucleotide
aggagcggccgcttaacactctcccctgttgaagctc

SEQ ID NO: 5 - DNA, 45 bp, Unknown
  oligonucleotide
tctggtactagtggccaggccggccttgagggtggtggctctgag

SEQ ID NO: 6 - DNA, 42 bp, Unknown
  oligonucleotide
caatagaaaattcatatggtttaccagcgccaaagacaaaag

SEQ ID NO: 7 - DNA, 25 bp, Unknown
  oligonucleotide
ggccctaggctgcctggtcaaggac

SEQ ID NO: 8 - DNA, 25 bp, Unknown
  oligonucleotide
ccggcctggccactagttttgtcac

SEQ ID NO: 9 - DNA, 30 bp, Unknown
  oligonucleotide
gttgaaatccggaactgcctctgttgtgtg

SEQ ID NO: 10 - DNA, 30 bp, Unknown
  oligonucleotide
ccttaattatatctagtgcggccgcttaac

SEQ ID NO: 11 - DNA, 4896 bp, Unknown
  vector
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgtacccgataaaagcggcttcctgacaggaggccgttttgttttgcagcccacctagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaattgaattcaaggagttaattatgaaaaaaaccgcgattgcgattgcggtggcgctggcgggctttgcgaccgtggcccaggcggcctctagataactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagcggccgcactagatataattaaggagataaatatgaaatatctgctgccgaccgcggcggcgggcctgctgctgctggcggcgcagccggcgatggcgctcgagctgatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggcccatcggtcttccccctggcaccctcctccaagagcacctctggcggcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccatccagcagcttgggcacccagacctacatctgcaacgtgaatcacaagcccagcaacaccaaggtggacaagaaagttgagcccaaatcttgtgacaaaactagtggccaggccggccttgagggtggtggctctgagggtggcggttctgagggtggcggctctgagggtggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgacggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaagagtcttaagctagctaagcggccggccgcattaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 12 - protein, 286 aa, Unknown
  beta lactamase sequence
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGVFIADKSGAGERGSRGIIAALGPDGKPSRIVVIYTTGSQATHDERNRQIAEIGASLIKHW

SEQ ID NO: 13 - protein, 22 aa, Unknown
  Omp sequence
MKKTAIAIAVALAGFATVAQAA

SEQ ID NO: 14 - protein, 13 aa, Unknown
  Kappa light chain constant region sequence
SSPVTKSFNRGEC

SEQ ID NO: 15 - protein, 22 aa, Unknown
  Pel B sequence
MKYLLPTAAAGLLLLAAQPAMA

SEQ ID NO: 16 - protein, 285 aa, Unknown
  human IgG CHI and partial hinge region
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGQAGLEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 17 - DNA, 5059 bp, Unknown
  vector PAX243mG1K
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgtacccgataaaagcggcttcctgacaggaggccgttttgttttgcagcccacctagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaattgaattcaaggagttaattatgaaaaaaaccgcgattgcgattgcggtggcgctggcgggctttgcgaccgtggcccaggcggcctctagataactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctccggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaagcggccgcactagatataattaaggagataaatatgaaatatctgctgccgaccgcggcggcgggcctgctgctgctggcggcgcagccggcgatggcgctcgagctgatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccctaggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtactagtggccaggccggccttgagggtggtggctctgagggtggcggttctgagggtggcggctctgagggtggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgacggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaagagtcttaagctagctaagcggccggccgcattaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 18 - protein, 286 aa, Unknown
  beta lactamase sequence
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 19 - protein, 22 aa, Unknown
  OmpA sequence
MKKTAIAIAVALAGFATVAQAA

SEQ ID NO: 20 - protein, 88 aa, Unknown
  murine Kappa LC constant region
SGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - protein, 22 aa, Unknown
  Pel B sequence
MKYLLPTAAAGLLLLAAQPAMA

SEQ ID NO: 22 - protein, 262 aa, Unknown
  Murine IgG1 CHI and partial hinge region
LGCLVKGYFPEPVTVTWNSGSLSSGVHTFPAVLQSDLYTLSSSVTVPSSTWPSETVTCNVAHPASSTKVDKKIVPRDCTSGQAGLEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 23 - DNA, 5059 bp, Unknown
  vector PAX313m/hK
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgtacccgataaaagcggcttcctgacaggaggccgttttgttttgcagcccacctagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaattgaattcaaggagttaattatgaaaaaaaccgcgattgcgattgcggtggcgctggcgggctttgcgaccgtggcccaggcggcctctagataactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagcggccgcactagatataattaaggagataaatatgaaatatctgctgccgaccgcggcggcgggcctgctgctgctggcggcgcagccggcgatggcgctcgagctgatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccctaggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtactagtggccaggccggccttgagggtggtggctctgagggtggcggttctgagggtggcggctctgagggtggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgacggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaagagtcttaagctagctaagcggccggccgcattaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 24 - protein, 286 aa, Unknown; identical to SEQ ID NO: 18
  beta lactamase sequence

SEQ ID NO: 25 - protein, 22 aa, Unknown
  OmpA sequence
MKKTAIAIAVALAGFATVAQAA

SEQ ID NO: 26 - protein, 67 aa, Unknown
  human Kappa LC constant region
SGTASVVCLLNNFYPREAKVQWKVDNALQSGNSQESVTEQDSKDSTYSLSSTLTLSKADYEKHKVYA

SEQ ID NO: 27 - protein, 43 aa, Unknown
  Pel B sequence
CEVTHQGLSSPVTKSFNRGECMKYLLPTAAAGLLLLAAQPAMA

SEQ ID NO: 28 - protein, 262 aa, Unknown; identical to SEQ ID NO: 22
  murine IgGICHI and partial hinge region

SEQ ID NO: 29 - DNA, 5068 bp, Unknown
  vector PAX313m/hG
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgtacccgataaaagcggcttcctgacaggaggccgttttgttttgcagcccacctagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaattgaattcaaggagttaattatgaaaaaaaccgcgattgcgattgcggtggcgctggcgggctttgcgaccgtggcccaggcggcctctagataactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctccggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaagcggccgcactagatataattaaggagataaatatgaaatatctgctgccgaccgcggcggcgggcctgctgctgctggcggcgcagccggcgatggcgctcgagctgatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccctaggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccatccagcagcttgggcacccagacctacatctgcaacgtgaatcacaagcccagcaacaccaaggtggacaagaaagttgagcccaaatcttgtgacaaaactagtggccaggccggccttgagggtggtggctctgagggtggcggttctgagggtggcggctctgagggtggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgacggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaagagtcttaagctagctaagcggccggccgcattaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 30 - protein, 286 aa, Unknown; identical to SEQ ID NO: 18
  beta lactamase sequence

SEQ ID NO: 31 - protein, 22 aa, Unknown
  OmpA sequence
MKKTAIAIAVALAGFATVAQAA

SEQ ID NO: 32 - protein, 88 aa, Unknown; identical to SEQ ID NO: 20
  murine Kappa LC constant region

SEQ ID NO: 33 - protein, 22 aa, Unknown
  Pel B sequence
MKYLLPTAAAGLLLLAAQPAMA

SEQ ID NO: 34 - protein, 265 aa, Unknown
  human IgGCHI and partial hinge region
LGCLVKDYFPEPVTVSWNSGALTSGVHTFPAVLQSSGLYSLSSVVTVPSSSLGTQTYICNVNHKPSNTKVDKKVEPKSCDKTSGQAGLEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 35 - DNA, 5068 bp, Unknown
  vector PAX313m/hGK
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgtacccgataaaagcggcttcctgacaggaggccgttttgttttgcagcccacctagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaattgaattcaaggagttaattatgaaaaaaaccgcgattgcgattgcggtggcgctggcgggctttgcgaccgtggcccaggcggcctctagataactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagcggccgcactagatataattaaggagataaatatgaaatatctgctgccgaccgcggcggcgggcctgctgctgctggcggcgcagccggcgatggcgctcgagctgatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccctaggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccatccagcagcttgggcacccagacctacatctgcaacgtgaatcacaagcccagcaacaccaaggtggacaagaaagttgagcccaaatcttgtgacaaaactagtggccaggccggccttgagggtggtggctctgagggtggcggttctgagggtggcggctctgagggtggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgacggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaagagtcttaagctagctaagcggccggccgcattaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 36 - protein, 286 aa, Unknown; identical to SEQ ID NO: 18
  beta lactamase sequence

SEQ ID NO: 37 - protein, 22 aa, Unknown
  OmpA sequence
MKKTAIAIAVALAGFATVAQAA

SEQ ID NO: 38 - protein, 88 aa, Unknown
  human Kappa LC constant region
SGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 22 aa, Unknown
  Pel B sequence
MKYLLPTAAAGLLLLAAQPAMA

SEQ ID NO: 40 - protein, 265 aa, Unknown; identical to SEQ ID NO: 34
  human IgG CHI and partial hinge region

(19) United States
(12) Patent Application Publication
(10) Pub. No.: US 2007/0009957 A1
(43) Pub. Date: Jan. 11, 2007

(54) Vectors used to create hybrid constant regions

(75) Inventors: Katherine S. Bowdish, Del Mar, CA (US); Shana Frederickson, Solana Beach, CA (US); Toshiaki Maruyama, La Jolla, CA (US)

Correspondence Address: FISH & NEAVE IP GROUP;ROPES & GRAY LLP, ONE INTERNATIONAL PLACE, BOSTON, MA, 02110-2624, US

(73) Assignee: Alexion Pharmaceuticals, Inc., Cheshire, CT (US)

(21) Appl. No.: 10/547,275

(22) PCT Filed: Mar. 4, 2004
(86) PCT No.: PCT/US04/06571

§ 371 Date: May 30, 2006

(30) Foreign Application Priority Data
Mar. 4, 2003 (US) 60451820

(51) Int. Cl. C40B 40/10; G01N 33/53; C07H 21/04; C12P 21/06; C07K 16/44; C12N 5/06

(52) U.S. Cl. 435/7.1; 435/69.1; 435/328; 435/320.1; 530/387.3; 536/23.53

(57) ABSTRACT

Expression vectors used for expression of mouse Fab libraries and expression of individual clones have portions of mouse constant regions replaced with human constant regions while maintaining desired cloning sites.

### RELATED APPLICATIONS This application claims priority to U.S. Provisional Application No. 60/451,820 filed Mar. 4, 2003, the disclosure of which is incorporated herein by reference.

### TECHNICAL FIELD

The disclosure relates to expression vectors encoding antibodies having hybrid constant regions.

### BACKGROUND

Plasmids are extrachromosomal genetic elements that are capable of autonomous replication within their hosts. Bacterial plasmids range in size from 1 Kb to 200 Kb or more and encode a variety of useful properties. Plasmid encoded traits include resistance to antibiotics, production of antibiotics, degradation of complex organic molecules, production of bacteriocins, such as colicins, production of enterotoxins, and production of DNA restriction and modification enzymes. Although plasmids have been studied for a number of years in their own right, particularly in terms of their replication, transmissibility, structure and evolution, with the advent of genetic engineering technology the focus of plasmid research has turned to the use of plasmids as vector for the cloning and expression of foreign genetic information.

Expression vectors are characterized by their ability not only to replicate the inserted foreign genetic information but also to promote the transcription of the genetic information into mRNA and its subsequent translation into protein. This expression requires a variety of regulatory genetic sequences including but not necessarily limited to promoters, operators, transcription terminators, ribosomal binding sites and protein synthesis initiation and termination codons. These expression elements can be provided with the foreign DNA segment as parts thereof or can be integrated within the vector in a region adjacent to a restriction site so that when a foreign DNA segment is introduced into the vector it falls under the control of those elements to which it is now chemically joined.

Expression vectors can be employed to generate relatively large quantities of any protein encoded by the vector. To do so, a host cell is transfected with the expression vector. As those skilled in the art will appreciate, the level of expression of the protein depends on a large number of factors, including but not limited to the compatibility between the host cell and the original source of the protein. For example, expression of murine antibodies in E-coli (one commonly used host cell) can be relatively poor compared, for example, to the level of expression of a similar human-derived antibody. The level of expression is particularly important where screening of an antibody library is being conducted. Those members of the library that are expressed in relatively low quantities are less likely to be identified in a panning process. Thus, potentially important or useful members of the library may be overlooked due to poor expression levels.

It would be advantageous to provide an expression vector that results in higher levels of expression for a greater number of members of an antibody library.

### SUMMARY

Expression vectors used for mouse Fab libraries and expression of individual clones typically have mouse kappa light chain and heavy chain constant regions with native restriction enzyme sites for direct cloning of mouse antibody genes. However, the level of expression of these Fab fragments in E. coli is less than optimal. By replacing portions of mouse constant regions with human constant regions in accordance with the present disclosure while maintaining the desired cloning sites, expression of mouse Fabs is greatly increased. In particularly useful embodiments, mouse Fab vectors contain partial human heavy chain constant region CH1 domain plus a partial human hinge region or partial human kappa chain constant region or both.

In another aspect a method of improving expression of antibodies is provided in which the antibodies are encoded by an expression vector that encode a hybrid constant region.

In another aspect, antibodies including a hybrid constant region are described.

In yet another aspect, a library of antibodies produced using an expression vector that encodes a hybrid constant region is described.

In yet another embodiment, methods of screening an antibody library are described wherein the library is prepared using an expression vector that encode a hybrid constant region.

### BRIEF DESCRIPTION OF THE DRAWINGS

FIGS. 1A-E show the nucleic acid sequence of the vector pAX243hgK (SEQ. ID NO: 11) and amino acid sequences thereby (SEQ. ID NOS: 12-16).

FIGS. 2A-G show the nucleic acid sequence of the vector pAX243mG1K (SEQ. ID NO: 17) and amino acid sequences encoded thereby (SEQ. ID NOS: 18-22).

FIGS. 3A-D show the nucleic acid sequence of the vector pAX313m/hK (SEQ. ID NO: 23) and amino acid sequences encoded thereby (SEQ. ID NOS: 24-28).

FIGS. 4A-D show the nucleic acid sequence of the vector pAX313m/hG (SEQ. ID NO: 29) and amino acid sequences encoded thereby (SEQ. ID NOS: 30-34).

FIGS. 5A-F show the nucleic acid sequence of the vector pAX313m/hGK (SEQ. ID NO: 35) and amino acid sequences encoded thereby (SEQ. ID NOS: 36-40).

FIG. 6 compares the expression of mouse Fab in hybrid vectors in accordance with this disclosure.

FIGS. 7A and B show the results of immunoblotting used to determine expression of mouse Fab in hybrid vectors in accordance with this disclosure.

FIG. 8 compares the expression of a mouse anti-PDGF Fab in hybrid vectors in accordance with this disclosure.

FIG. 9 compares the binding of a mouse anti-PDGF Fab in hybrid vectors in accordance with this disclosure.

FIG. 10 shows the Fab expression of a library made in PAX1 33m/hG vector before panning.

FIG. 11 shows the Fab expression binding to FLJ32028-Fc and mouse IgG Fc of IgG1 kappa clones in PAX313,/hG vector after 4 rounds of panning on FLJ32028-Fc.

### DETAILED DESCRIPTION

Expression vectors used for mouse Fab libraries and expression of individual clones encode a hybrid antibody constant region that includes mouse constant regions with human constant regions while maintaining desired cloning sites. Use of such expression vectors greatly increases expression of mouse Fabs compared to the expression of mouse Fabs that include a fully mouse constant region. The amount of mouse constant region that is replaced with human constant region is an amount sufficient to increase expression of antibodies encoded by the vector. The mouse constant region that is replaced with human constant region can be in the heavy chain constant region, the light chain constant region, or both. In particularly useful embodiments, mouse Fab vectors contain partial human heavy chain constant region CH1 domain plus a partial human hinge region or partial human kappa chain constant region or both.

Antibodies including a hybrid constant region are produced when the vectors are transfected into a host cell. Techniques for transfection are within the purview of those skilled in the art. In particularly useful embodiments, the host cell is E. Coli.

The vectors described herein can also be used to prepare antibody libraries wherein each member of the library includes a hybrid constant region. The resulting antibody library prepared using an expression vector that encodes a hybrid constant region in accordance with this disclosure can be screened using techniques known to those skilled in the art to identify antibodies having desired characteristics, such as, for example, binding affinity.

While the following examples are described with respect to a series of particular steps for producing expression vectors encoding a hybrid antibody constant region and for using the expression vector for expression of particular antibodies, it should of course be understood that the present disclosure is not limited to this particular sequence of steps or the particular antibodies produced. Those skilled in the art will readily envision other schemes for preparing expression vectors in accordance with this disclosure. Those skilled in the art will also readily appreciate that antibodies other than those specifically exemplified below can be ligated into the expression vectors described herein. Techniques for doing so are within the purview of those skilled in the art.

### EXAMPLE I

Construction of PAX313m/hG

The vector PAX243hGK is prepared from PAX131, a phagemid vector described in Published International Patent Application No. WO 02/088315 A2 published Nov. 7, 2002, the disclosure of which is incorporated herein by this reference. PAX131 was first digested with Not I (NEB) and filled-in with Klenow fragment (NEB). Blunt-end ligation was performed with T4 DNA ligase and electroporated into TOP10F′ cells. Individual clones were checked for the absence of Not I site by Not I digestion. Next, EcoSpe oligo (5′ AAT TCA AGG AGT TAA TTA TGA AAA AAA CCG CGA TTG CGA TTG CGG TGG CGC TGG CGG GCT TTG CGA CCG TGG CCC AGG CGG CCT CTA GAA TCT GCG GCC GCA 3′- SEQ. ID NO: 1) and SpeEco oligo (5′ CTA GTG CGG CCG CAG ATT CTA GAG GCC GCC TGG GCC ACG GTC GCA AAG CCC GCC AGC GCC ACC GCA ATC GCA ATC GCG GTT TTT TTC ATA ATT AAC TCC TTG 3′-SEQ. ID NO: 2) were hybridized and ligated to EcoR I/Spe I digested PAX131 Not I (−) vector obtained above. Inserted EcoSpe duplex oligo contains a Not I site for subsequent cloning purposes. Then, the kappa light chain constant region was amplified by PCR amplification. CK Xba I primer (5′ GGA GTC TAG ATA ACT GTG GCT GCA CCA TCT GTC TTC 3′- SEQ. ID NO: 3) and CK Not I primer (5′ AGG AGC GGC CGC TTA ACA CTC TCC CCT GTT GAA GCTC 3′-SEQ. ID NO: 4) were used. The amplified product and PAX131 Not I (−) vector were digested with Xba I/Not I and ligated. The ligated product was electroporated into TOP10F′ cells and individual clones were sequenced. A clone with no PCR error (PAX131K) was chosen for further modification.

For better Fab expression truncated gene III was amplified from PAX131K by PCR amplification. Tg3SpeSfi primer (5′ TCT GGT ACT AGT GGC CAG GCC GGC CTT GAG GGT GGT GGC TCT GAG 3′- SEQ. ID NO: 5) and tg3Nde primer (5′ CAA TAG AAA ATT CAT ATG GTT TAC CAG CGC CAA AGA CAA AAG 3′-SEQ. ID NO: 6) were used. The amplified product and PAX131K were digested with Xba I/Nde I and ligated. The ligated product was electroporated into TOP10F′ cells and individual clones were sequenced. A clone with no PCR error (PAX243K) was chosen for further modification. Human IgG CH1 and partial hinge region gene was ordered from Aptagen. The gene fragment and PAX243K vector were digested with Xho I/Spe I and ligated. The ligated product was electroporated into TOP10F′ cells and individual clones were checked for the insertion of the fragment. The resultant vector was named PAX243hGK-int. Finally for cloning efficiency, an 815 bp stuffer fragment was inserted into PAX243hGK-int vector by Xho I/Apa I digestion and ligation. The final vector was named PAX243hGK.

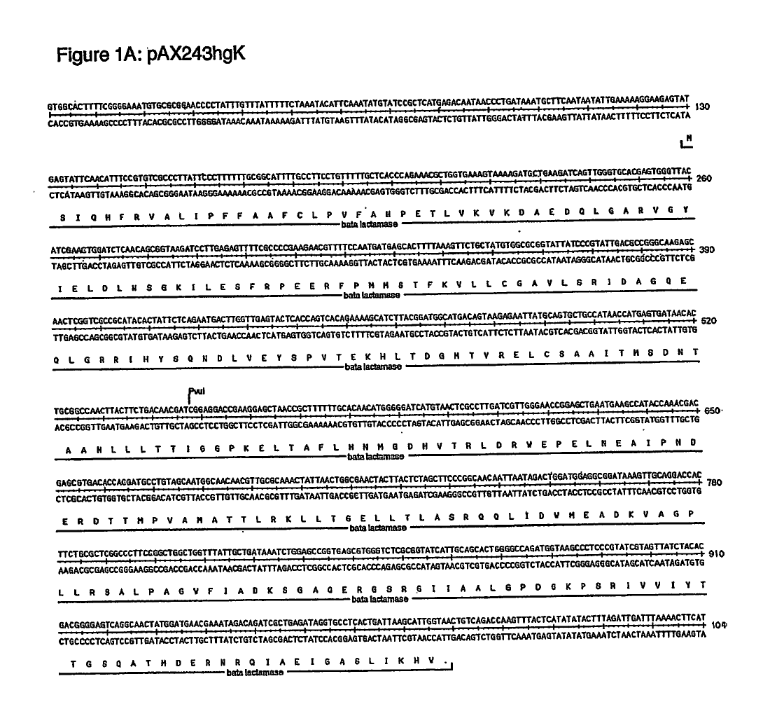

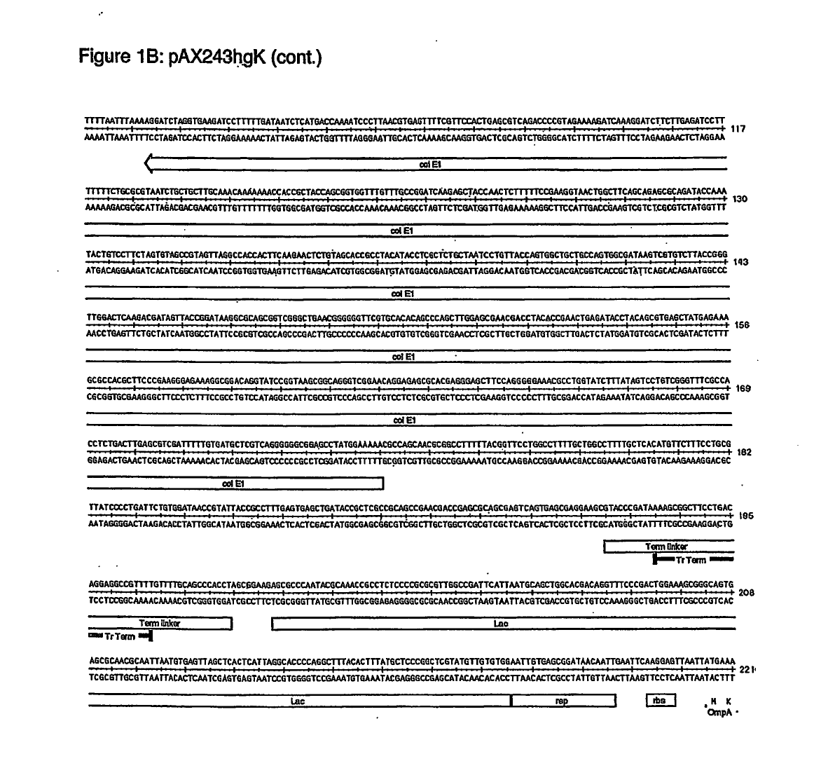

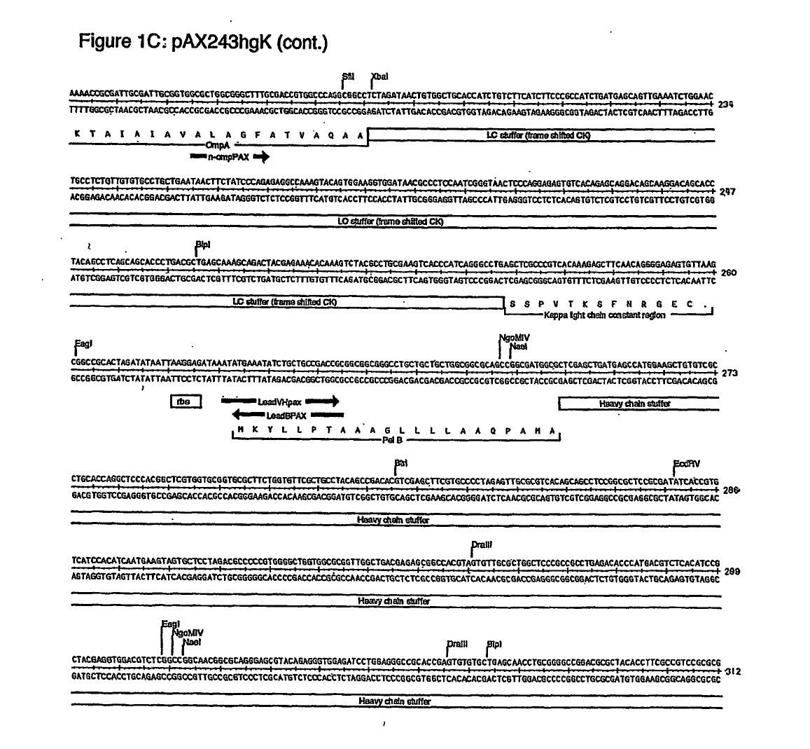

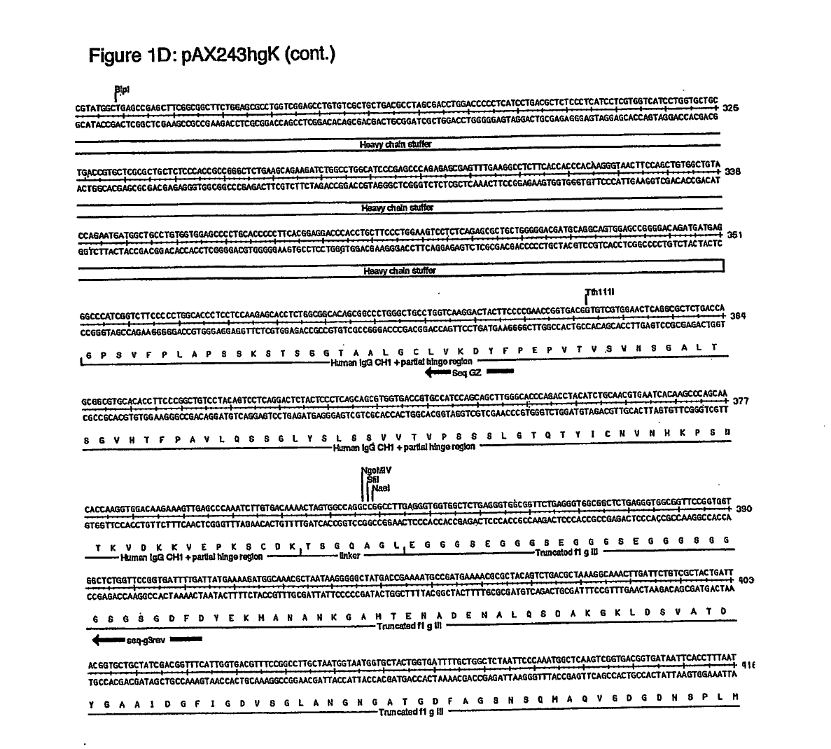

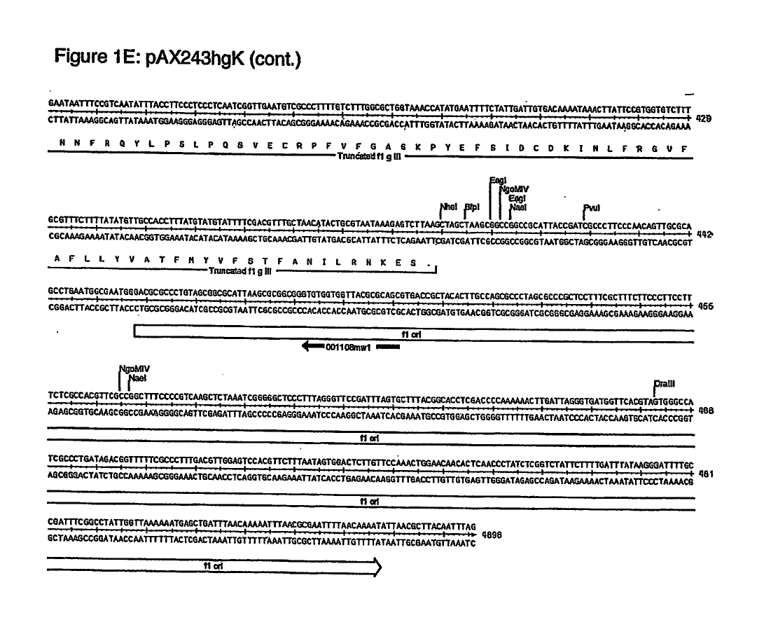

For the construction of mouse Fab vector with partial human heavy chain, human constant region CH1 plus partial human hinge region, PCR amplification was performed to obtain a fragment from the portion that corresponds to Bln I site in the mouse heavy chain constant region and to the end portion that extends into a partial hinge region with Spe I site in the human Fab vector PAX243hGK. (FIG. 1A-E). The primers used are:

BlnIhCH1 5′ GGCCCTAGGCTGCCTGGTCAAGGAC 3′ (SEQ. ID NO: 7); and

SpelhCH1 5′ CCGGCCTGGCCACTAGTTTTGTCAC 3′ (SEQ. ID NO: 8).

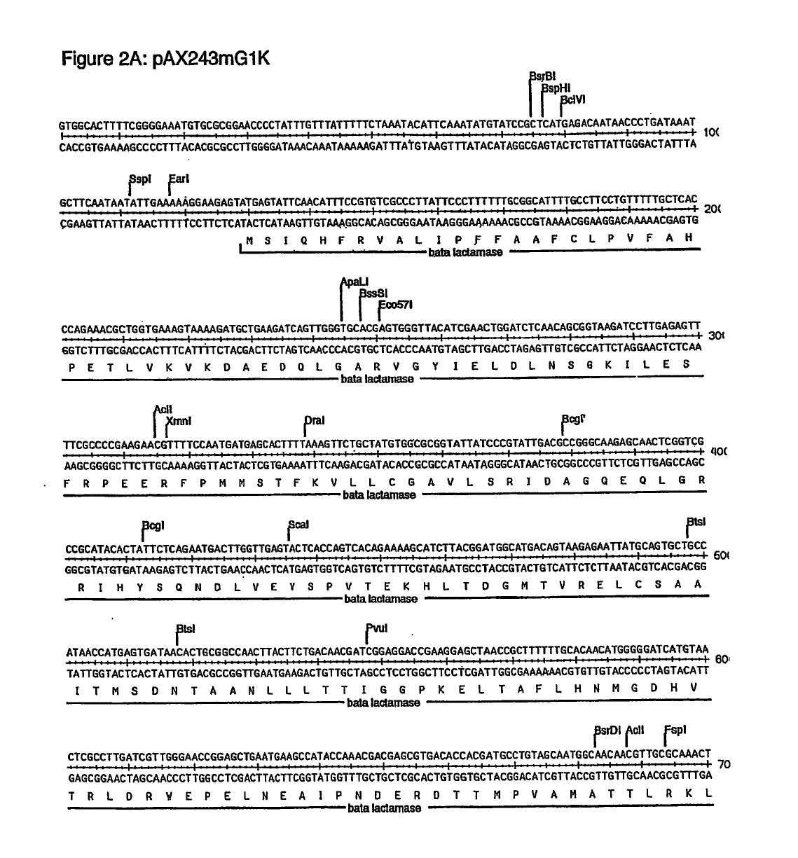

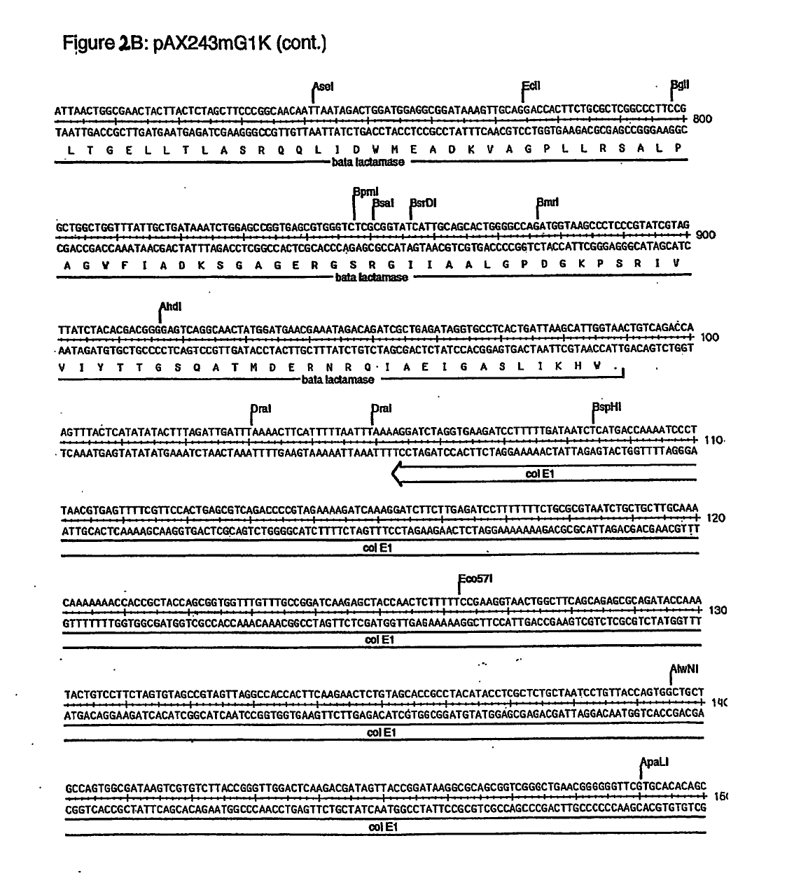

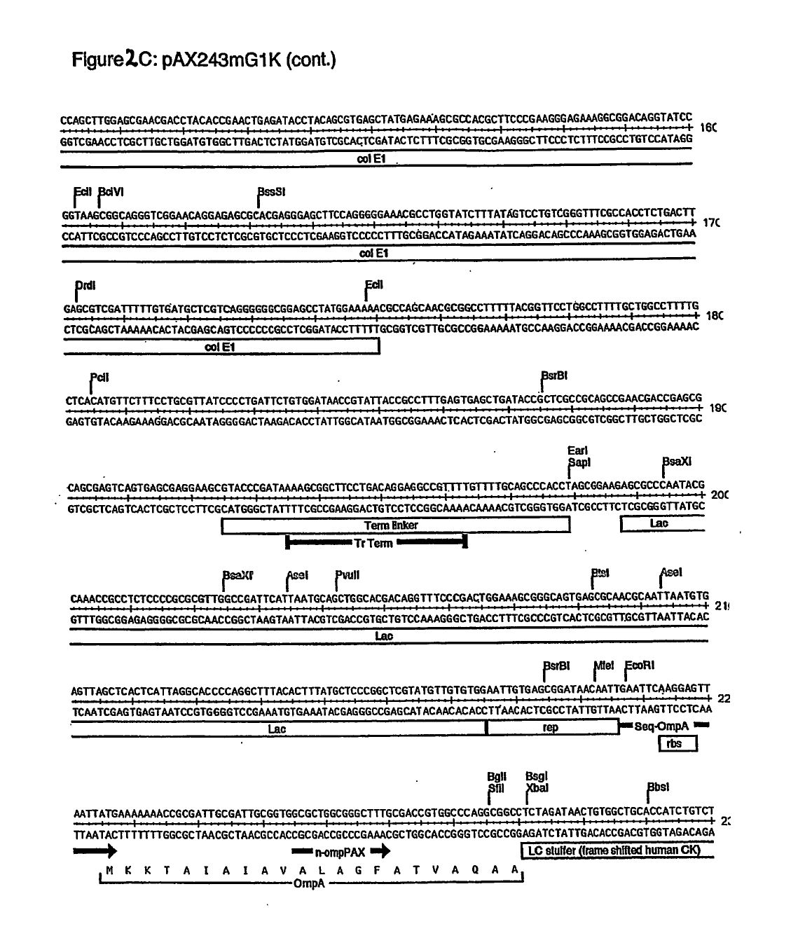

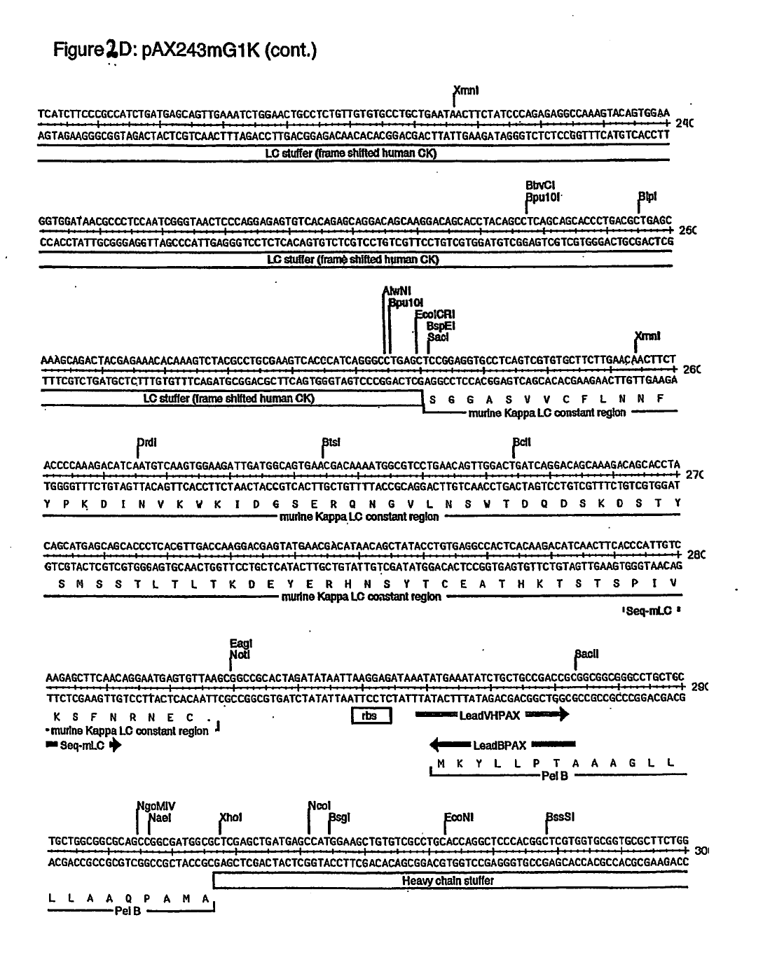

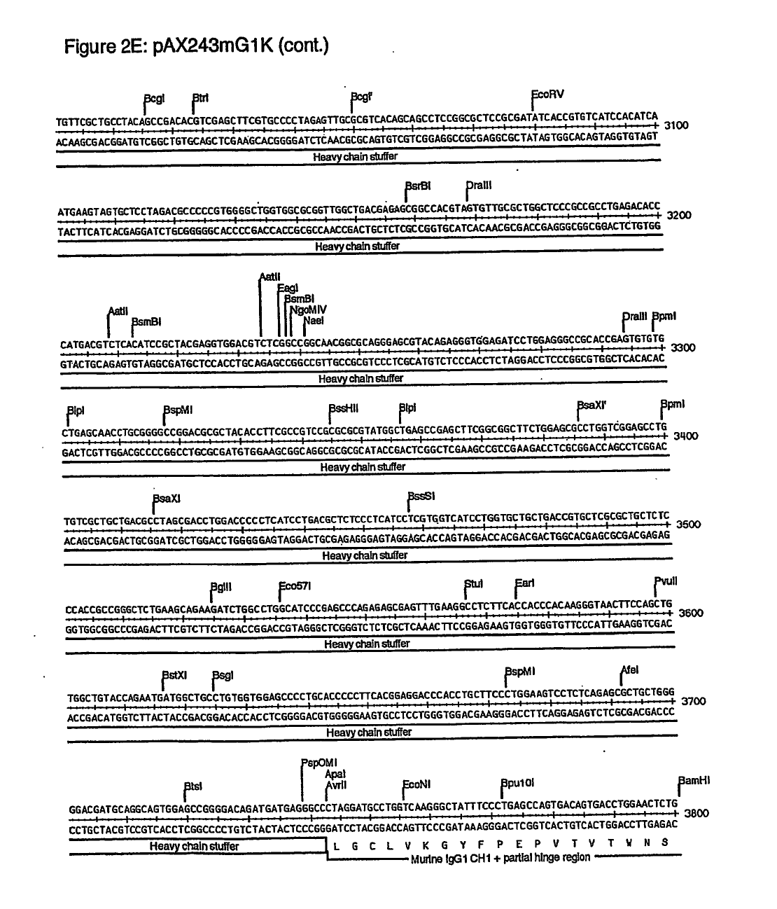

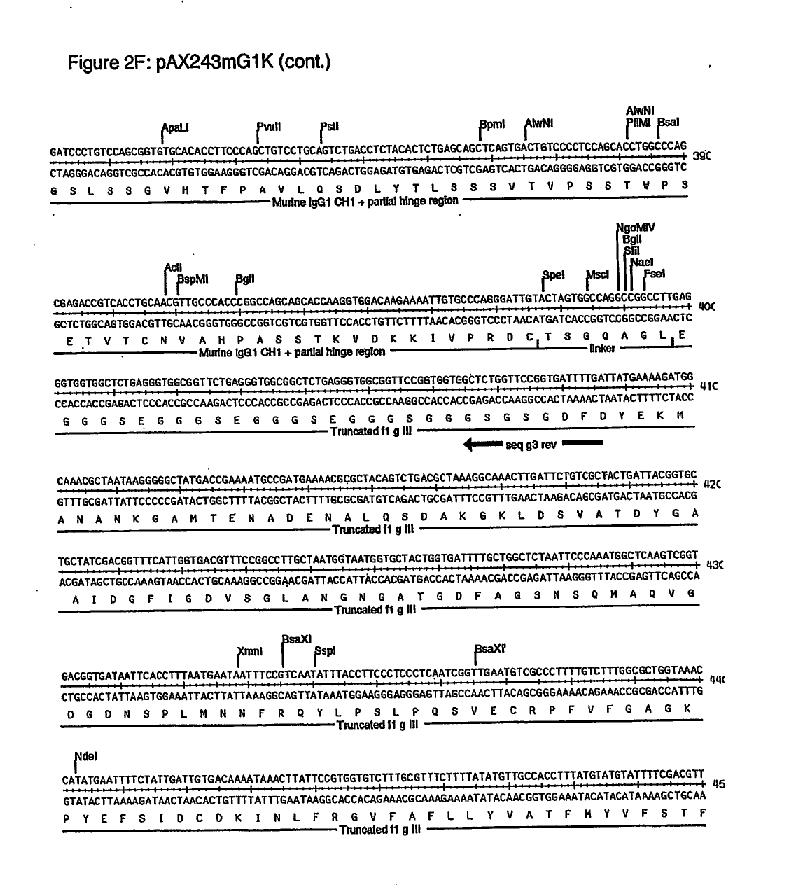

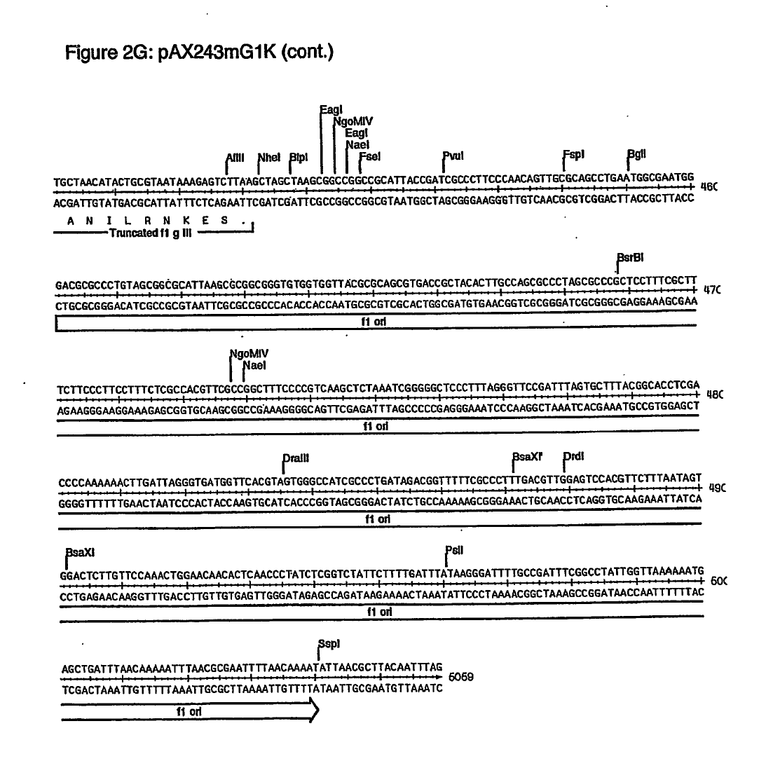

PCR amplification was performed using Advantage HF polymerase mix kit (BD Biosciences) for 30 cycles. BlnhCH1 contains Bln I site and SpelhCH1 contains Spe I site both, for the purpose of cloning at the respective cloning positions in the mouse Fab vector. Amplified product was run on a 1% agarose gel (Invitrogen Life Technologies) and 264 bp fragment was collected and purified using QIAGEN Gel Extraction Kit. The purified product was, then, digested with Bln I and Spe I. A 244 bp fragment was collected from 1% agarose gel and purified with QIAGEN Gel Extraction Kit. The mouse Fab vector PAX243mGIK (FIG. 2) was also digested with Bin I and Spe I. A 4824 bp fragment was collected on 0.6% agarose gel and purified with QIAGEN Gel Extraction Kit. These fragments were ligated and the reaction was electroporated into TOP10F′ E. coli cells.

Single colonies were grown in 1 ml SB medium with 50 μg/ml carbenicillin and 20 mM glucose and plasmid DNA was prepared with QIAGEN mini prep columns. Insertion of human heavy chain constant region was determined by the novo presence of Age I (PinA I) site in the human heavy chain constant region that is not present in the original mouse heavy chain. Positive DNA clones were sent for DNA sequencing analysis (Retrogen Inc., San Diego, Calif., USA) to check for the presence of PCR errors. Ten nanograms of DNA that had no PCR error was re-transformed in TOP10F′ cells and a single colony was grown in 10 ml SB medium with 50 μg/ml carbenicillin and 20 mM glucose for approximately 6 hrs. The culture was then transferred to 500 ml SB medium containing 50 μg/ml carbenicillin and 20 mM glucose and grown overnight at 37° C. in an environmental shaker at 250 rpm. The culture was spun down and DNA was prepared by HiSpeed Maxi Prep (QIAGEN).

### EXAMPLE 2

Construction of PAX313m/hK

For the construction of mouse Fab vector with human kappa light chain, PCR amplification was performed to obtain a fragment from the portion that corresponds to BspE I site in the mouse kappa light chain constant region and to the end portion with Not I site in one of the human antibody clone in the human Fab vector PAX243hGK (FIG. 1A-E). The primers used are:

BspEIhCK 5′ GTTGAAATCCGGAACTGCCTCTGTTGTGTG 3′ (SEQ. ID NO: 9); and

NotIhCK-rev 5′ CCTTAATTATATCTAGTGCGGCCGCTTAAC 3′ (SEQ. ID NO: 10).

PCR amplification was performed using Advantage HF polymerase mix kit (BD Biosciences) for 30 cycles. The BspEIhCK contains BspE I site and NotIhCK-rev contains Not I site, both for the purpose of cloning at the respective cloning positions in the mouse Fab vector. Amplified product was run on a 1% agarose gel (Invitrogen Life Technologies) and 299 bp fragment was collected and purified using QIAGEN Gel Extraction Kit. The purified product was then digested with BspE I and Not I. A 272 bp fragment was collected from 1% agarose gel and purified with QIAGEN Gel Extraction Kit. The mouse Fab vector PAX243mGIK (FIG. 2A-G) was also digested with BspE I and Not I. From this digestion, a 4791 bp fragment was collected on 0.6% agarose gel and purified with QIAGEN Gel Extraction Kit. These fragments were ligated and the reaction was electroporated into TOP10F′ E. coli cells.

Single colonies were grown in 1 ml SB medium with 50 μg/ml carbenicillin and 20 mM glucose. Plasmid DNA was prepared with QIAGEN mini prep columns. Insertion of human kappa light chain constant region was determined by the additional Sac I site in the human kappa light chain constant region that is not present in the original mouse kappa light chain. Positive DNA clones were sent for DNA sequencing analysis (Retrogen) to check for the presence of PCR errors. Ten nanograms of DNA that had no PCR error was then re-transformed in TOP10F′ cells. A single colony was grown in 10 ml SB medium with 50 μg/ml carbenicillin and 20 mM glucose for approximately 6 hrs. The culture was transferred to 500 ml SB medium containing 50 μg/ml carbenicillin and 20 mM glucose and grown overnight at 37° C. in an environmental shaker at 250 rpm. The culture was spun down and DNA was prepared by HiSpeed Maxi Prep (QIAGEN).

### EXAMPLE 3

Construction of PAX313m/hGK

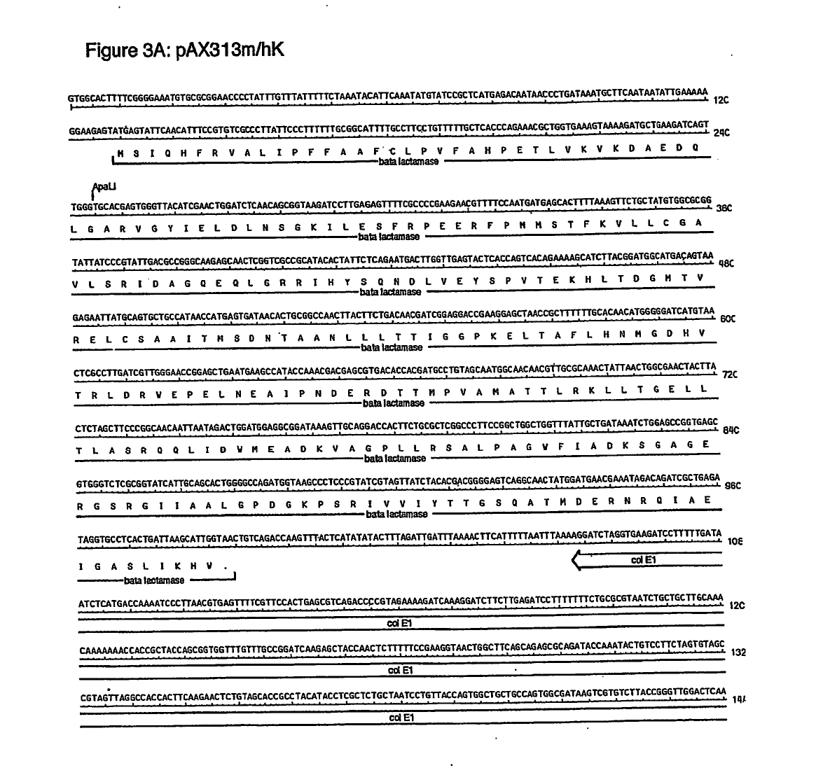

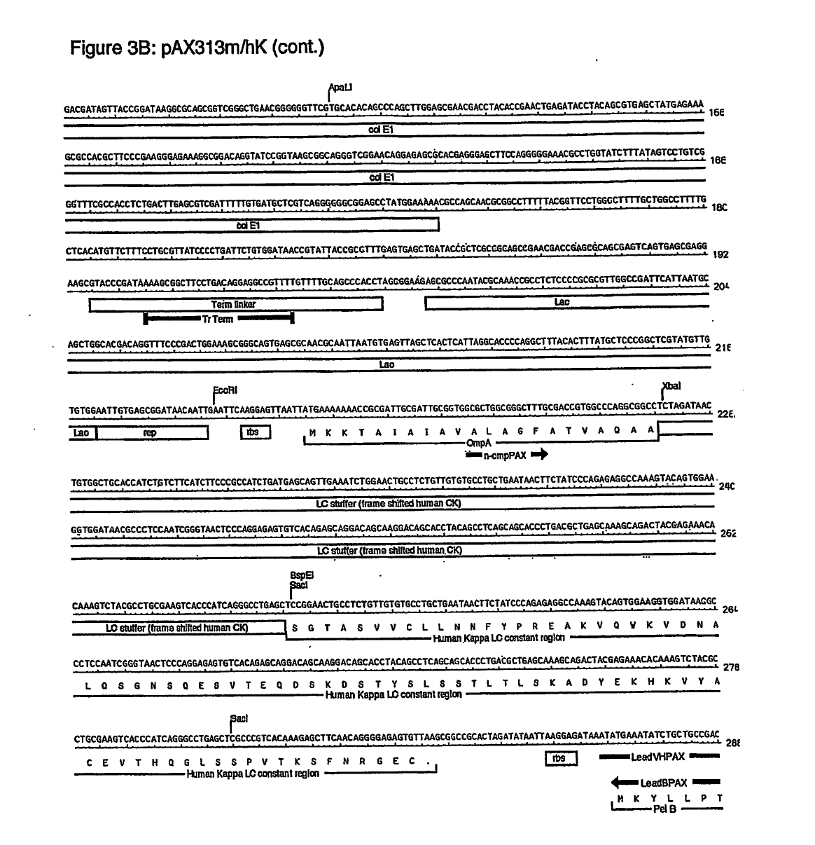

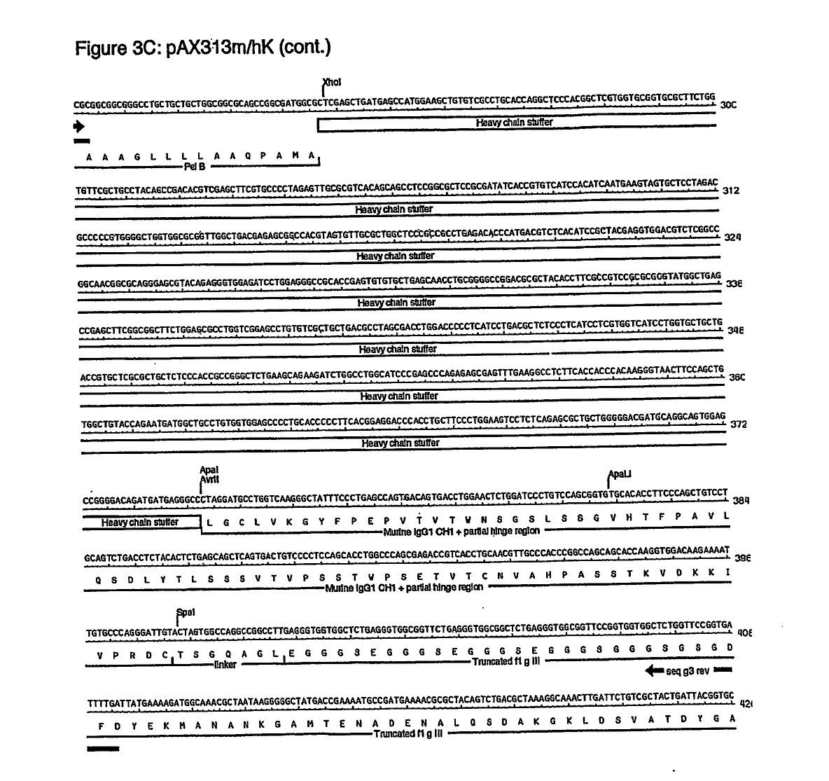

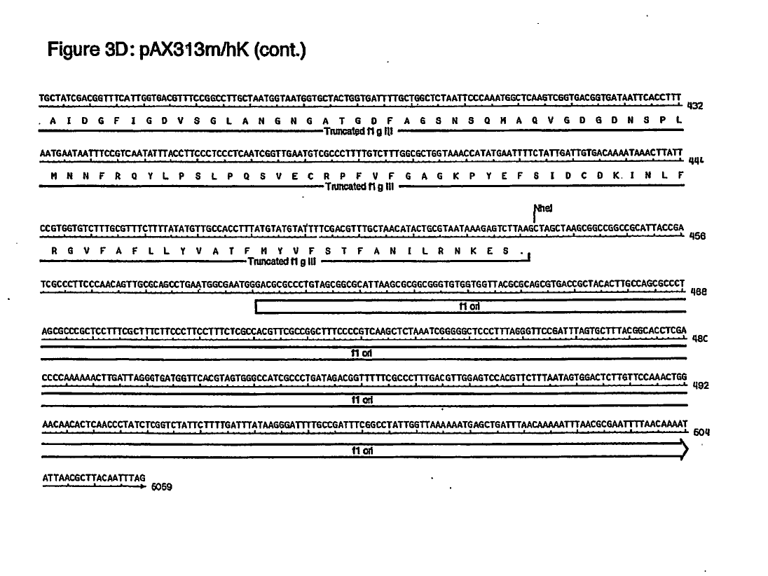

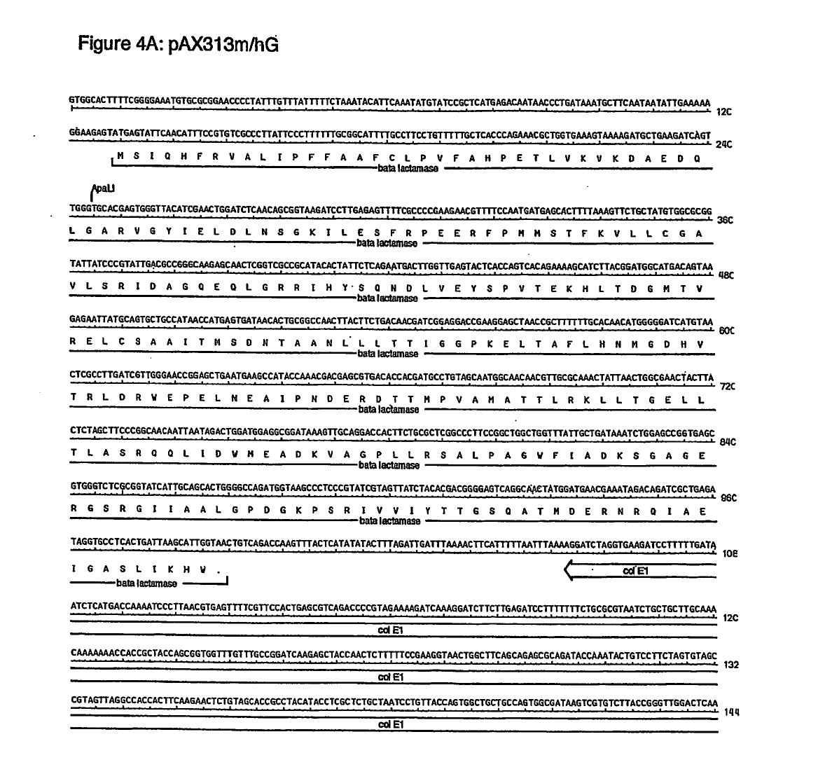

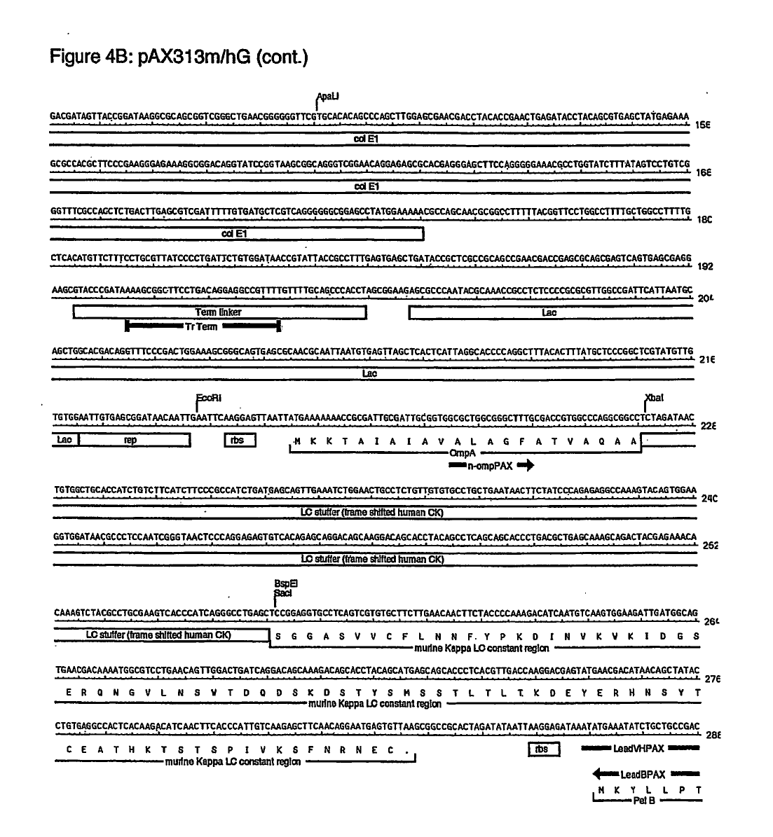

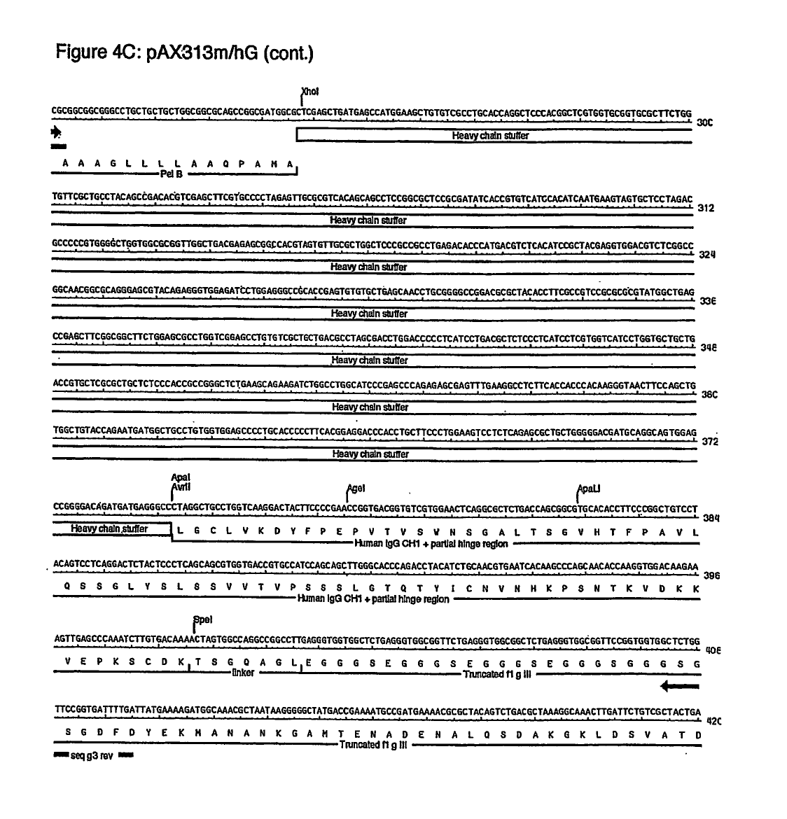

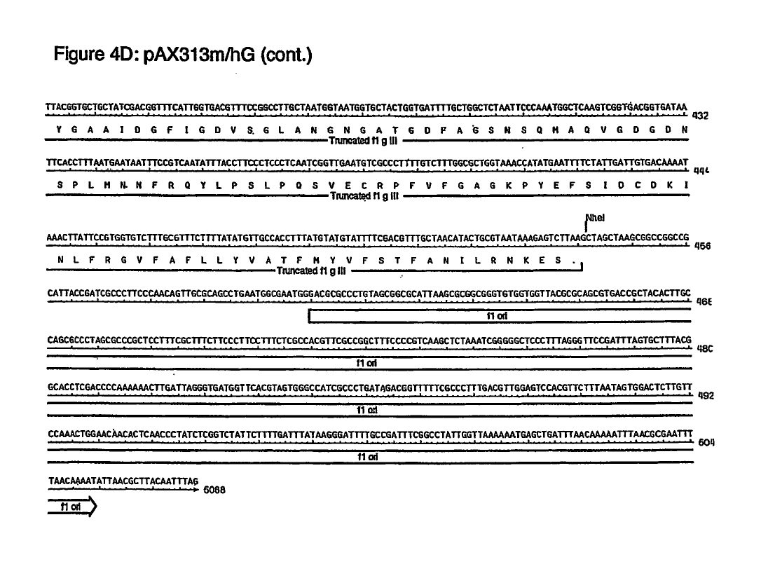

For the construction of mouse Fab vector with partial human heavy chain human constant region CH1, plus partial human hinge region and human kappa light chain, human kappa light chain constant region was transferred from PAX313m/hK (FIG. 3A-D) vector to PAX313m/hG (FIG. 4A-D) vector by digestion of both vector DNA with BspE I and Not I. A 4796 bp fragment from PAX313m/hG (FIG. 4A-D) and 272 bp fragment from PAX313m/hK (FIG. 3) were collected and purified from agarose gel using QIAGEN Gel Extraction Kit. These fragments were ligated and the reaction was electroporated into TOP10F′ E. coli cells.

Single colonies were grown in 1 ml SB medium with 50 μg/ml carbenicillin and 20 mM glucose. The plasmid DNA was prepared with QIAGEN mini prep columns. Insertion of human kappa light chain constant region into PAX313m/hG (FIG. 4A-D) vector was determined by the additional Sac I site in the human kappa light chain constant region that is not present in the original mouse kappa light chain in the PAX313m/hG (FIG. 4A-D) vector. Ten nanograms of DNA that had no PCR error was re-transformed in TOP10F′ cells. A single colony was grown in 10 ml SB medium with 50 μg/ml carbenicillin and 20 mM glucose for approximately 6 hrs. The culture was transferred to 500 ml SB medium containing 50 μg/ml carbenicillin and 20 mM glucose and grown overnight at 37° C. in an environmental shaker at 250 rpm. The culture was spun down and DNA was prepared by HiSpeed Maxi Prep (QIAGEN).

### EXAMPLE 4

Comparison of Fab Expression and/or Binding in Mouse/Human Hybrid Vectors

To determine the expression of mouse Fabs in these different mouse/human hybrid constructs, known mouse Fabs were expressed in these vectors. The Fab expression and/or bindings to the original antigens were then compared. Initially, mouse IgG1 kappa Fab specific to human IgE, that is known to express well, was used for this comparison. Both light chain and heavy chain fragments were digested at the corresponding cloning sites and sequentially cloned into each vector. After the final constructions were done, Fab expression ELISA was performed. Fab in each hybrid vector was grown in 1 ml SB medium in the presence of 50 μg/ml carbenicillin. Fab expression was induced with 1 mM IPTG and MI3 VCS helper phage at 30° C. overnight. The culture was spun down and the supernatant containing Fab was used for ELISA experiment. Microtiter wells were coated either with goat anti-human IgG F(ab′)₂ or rabbit anti mouse IgG F(ab′)₂ or mixture of both (Pierce) (4 μg/ml in PBS) at 4° C. overnight.

The wells were washed with PBS and blocked with 1% BSA/PBS 1 hr at 37° C. The blocker was flicked off. Fab containing supernatant was added and incubated for approximately 1.5 hrs. The wells were washed and bound Fab was detected either with alkaline phosphatase-conjugated goat anti-human IgG F(ab′)s or goat anti-mouse IgG F(ab′)₂ antibody (1:500 in 1% BSA/PBS) (Pierce).

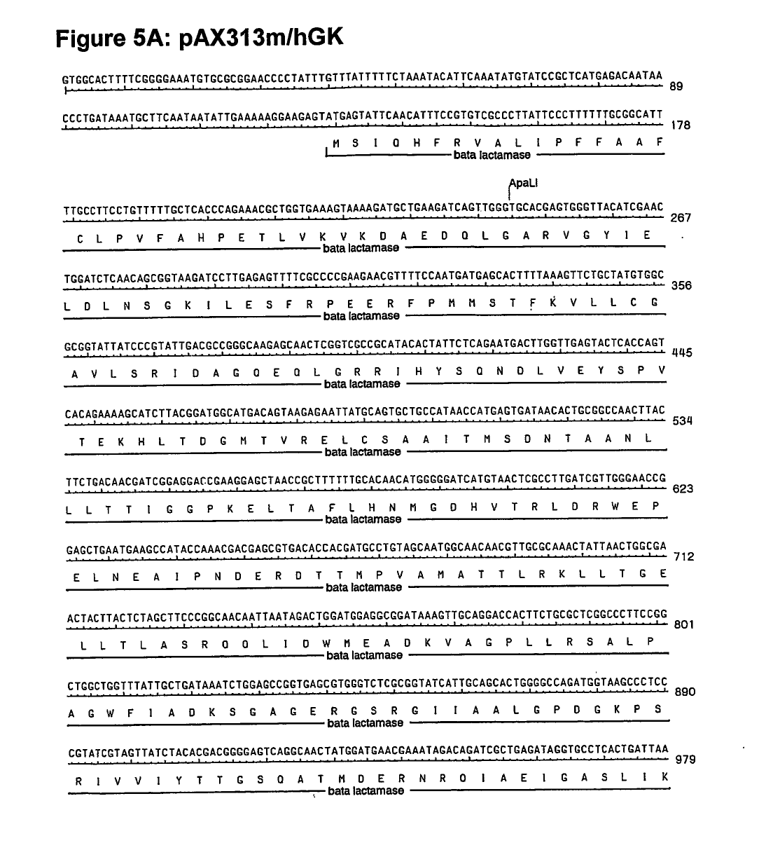

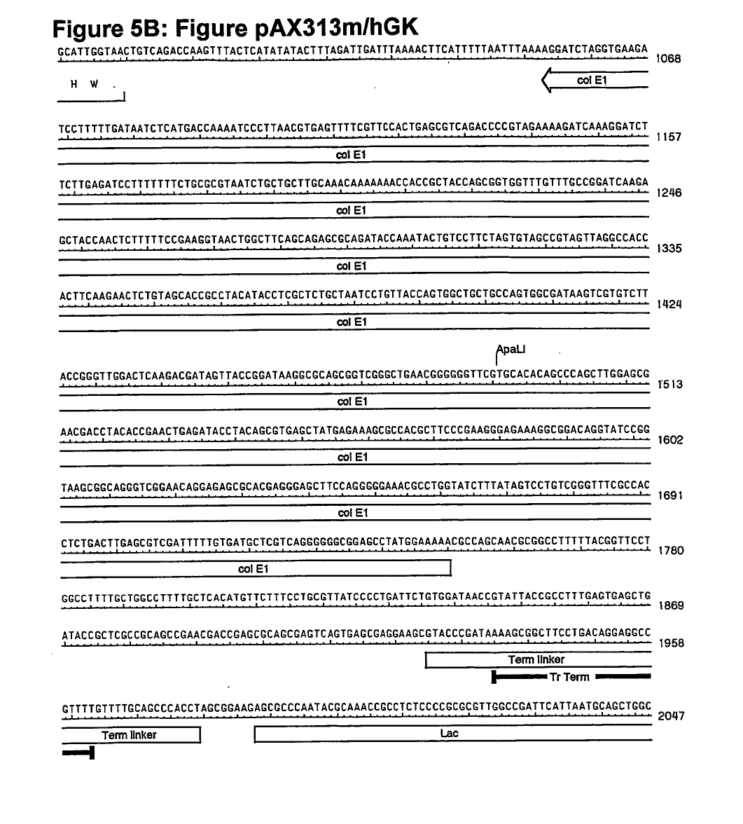

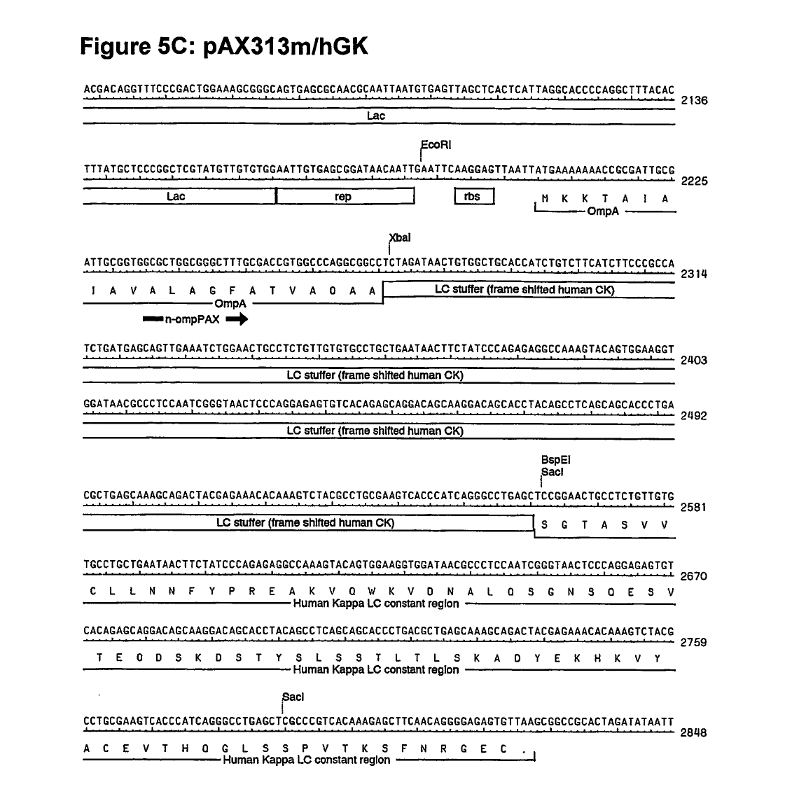

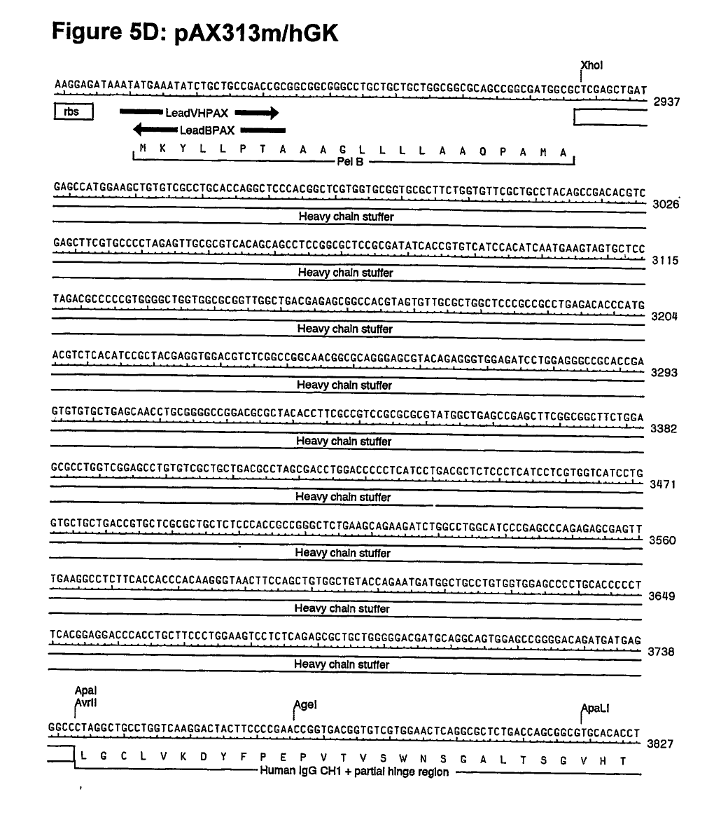

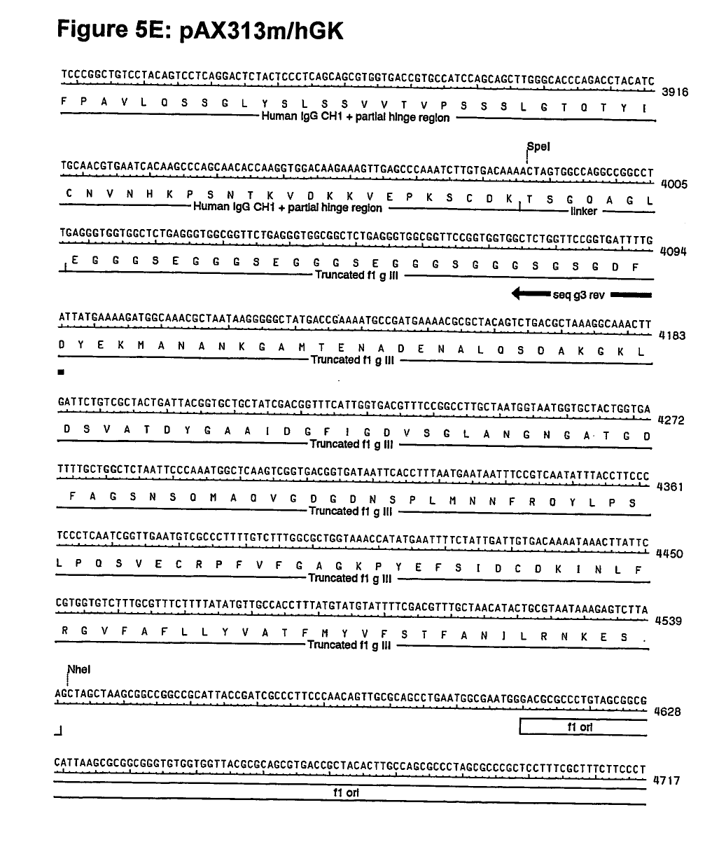

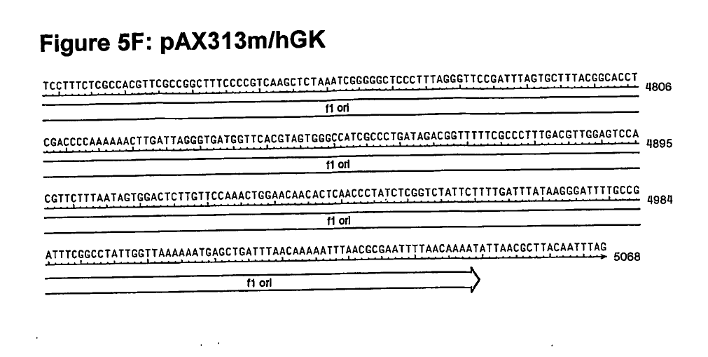

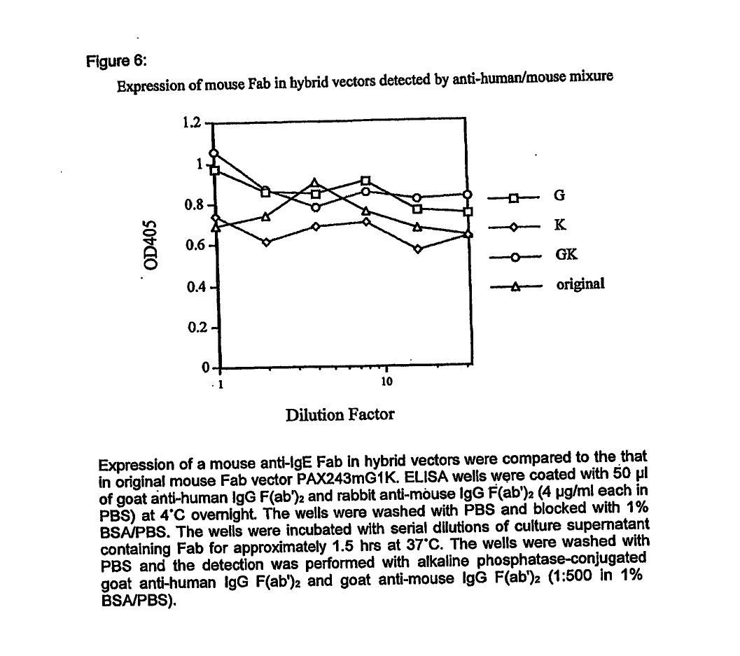

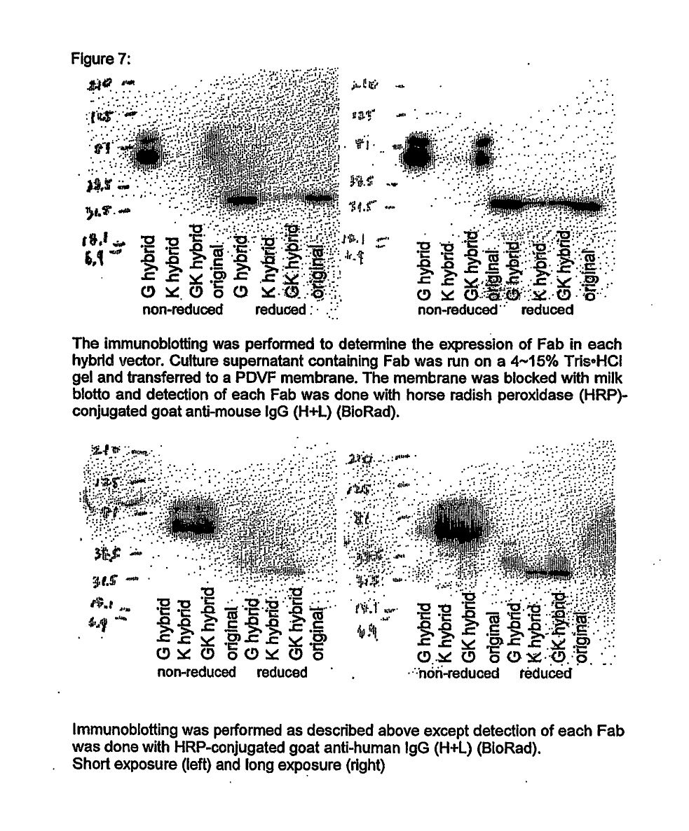

Compared with the Fab in the original mouse Fab vector PAX243mG1K, PAX313mh/G and PAX313m/hGK (see FIGS. 5A-F) showed the same or even better Fab expression. (See FIG. 6.) Western blot analysis also showed similar results. FIG. 7a shows detection of Fab in each Vector detected by goat anti-mouse IgG (H+L) by western blot analysis. Goat anti-mouse IgG (H+L) specifically detects mouse kappa light chain of Fab in original PAX243mG1K vector and PAX313m/hG hybrid vector. The expression of Fab in PAX313m/h G vector is better than that in PAX243mG1K vector. These data suggest that mouse kappa light chain constant region is expressed better when it is paired with mouse/human IgG1 heavy chain constant region hybrid. FIG. 7b shows detection of Fab in each vector detected by goat anti-human IgG (H+L) by western blot analysis. Goat anti-human IgG (H+L) specifically detects human kappa light chain of Fab in PAX313m/hGK hybrid vector and PAX313m/hK hybrid vector. The expression of Fab in PAX31 3m/hGK vector is better than that in PAX313m/hK vector. These data suggest that mouse/human kappa light chain constant region hybrid is not expressed very well when paired with mouse IgG1 heavy chain constant region but is expressed very well when it is paired further with mouse/human IgG1 heavy chain constant region hybrid.

For the next comparison, a mouse Fab, known to express poorly, that is specific to human PDGF was used for this comparison. Since this clone has different cloning sites from our PAX vectors, primers were designed to engineer correct cloning sites in light chain and heavy chain. After PCR amplification of kappa light chain and heavy chain, fragments were purified with digested with Xba I/BspE I and Xho I/Bln I sites, respectively.

Fragments were purified on 1% agarose gel and 391 bp fragments (kappa light chain) and 438 bp fragments (heavy chain) were purified with QIAGEN Gel Extraction Kit. These fragments were sequentially cloned into PAX243mG1K vector. Binding to specific antigen was performed as follows. The antigen human PDGF was coated at 4 μg/ml in 0.1 M NaHCO₃, pH8.6, at 4° C. overnight. The plates were washed with PBS and blocked with 1% BSA/PBS. Fab supernatant expressed, as described above, was incubated with antigen for approximately 1.5 hrs at 37° C. The wells were washed with PBS and bound Fab was detected with alkaline phosphatase-conjugated goat anti-mouse IgG F(ab′)₂ antibody (1:500 in 1% BSA/PBS) (Pierce). The clone that showed strongest binding were sent for DNA sequencing to check for PCR errors. The kappa light chain and heavy chain from clones that had no PCR errors were chosen and used for the construction of mouse/human hybrid Fabs. Kappa light chain and heavy chain were digested with Xba I/BspE I and Xho I/Bin I, respectively and sequentially cloned into each mouse/human hybrid vectors at corresponding sites.

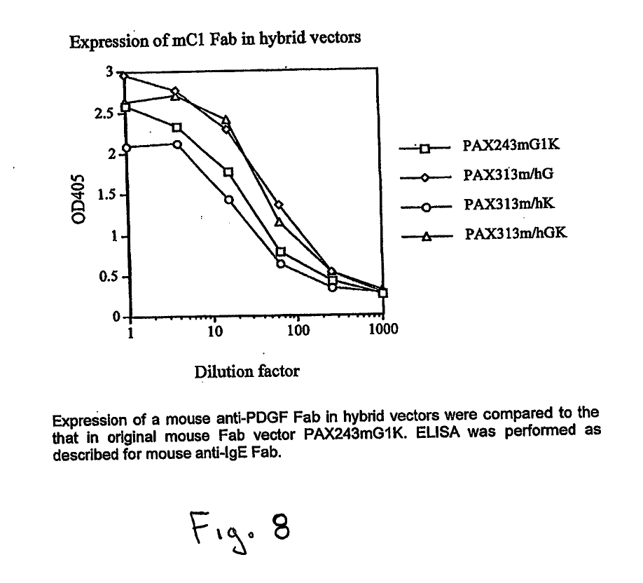

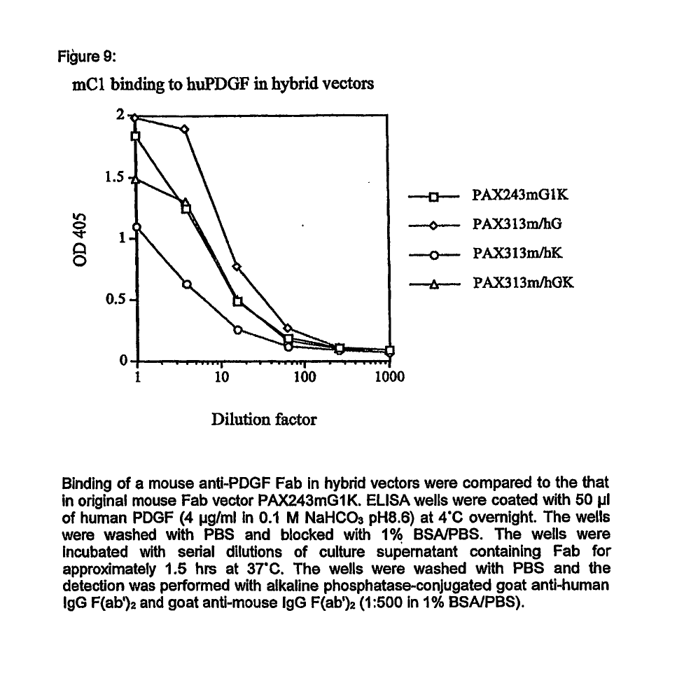

Fabs in each hybrid vectors and PAX243mG1 K were compared for Fab expression and binding to the original antigen as described above. The result shows that the expression of the Fab in either PAX313m/hG or PAX313m/hGK is at least 2-4 times better than in the original PAX243mG1 K vector. (See FIG. 8.) The trend was the same for binding to the specific antigen. (See FIG. 9.)

The present mouse/human hybrid vector constructs can be used for the de novo construction of mouse antibody libraries and for improving the existing mouse antibody libraries or Fabs that have expression problems as well. This can be accomplished by matching the 5′ end cloning sites of antibody fragments since 3′ end cloning utilizes native murine restriction sites in the kappa and heavy chain constant regions.

### EXAMPLE 5

Construction of a Fab Library in a Mouse/Human Hybrid Vector.

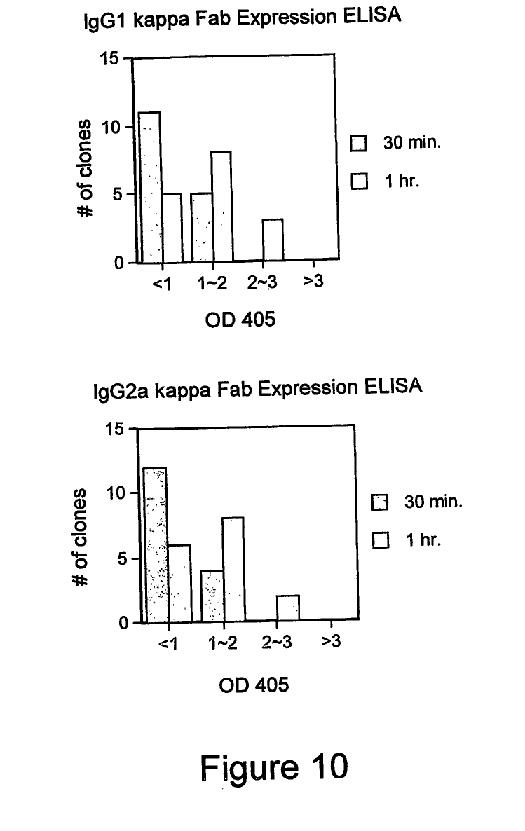

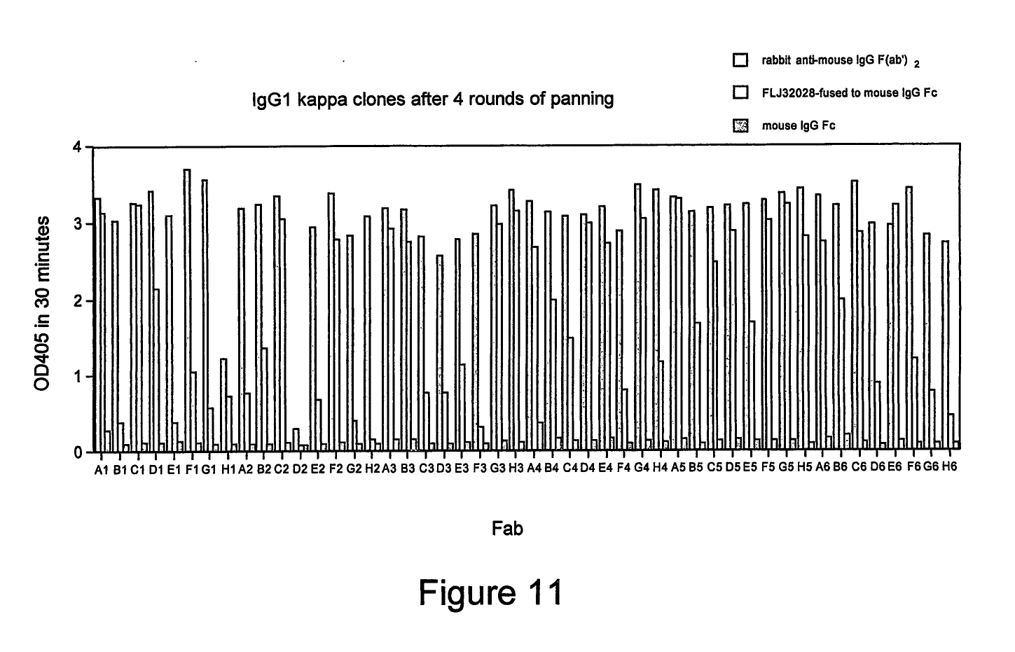

A Fab library was made from a spleen sample of a mouse immunized with FLJ32028 peptide in PAX313m/hG vector according to the method described in WO03/025202A2. Briefly, total RNA was extracted from the spleen sample and messenger RNA was purified using Oligotex kit (QIAGEN, Valencia, Calif.). First strand cDNA was synthesized using SUPERSCRIPT First-Strand Synthesis System for RT-PCR (Invitrogen Life Technologies, Carlsbad, Calif.) according to the manufacturer's protocol. First strand cDNA was digested with Hpa I for kappa light chain, Xcm I for IgG1 heavy chain, and BsaJ I for IgG2a heavy chain. 2^{nd} strand cDNA was synthesized with primers that anneals to the framework 1 region of each gene fragment and extension reaction was performed with oligos that hybridize to the constant region of each gene fragment. Following single primer amplification, each fragment was purified with PCR purification kit (QIAGEN) and digested with appropriate enzymes for cloning into the vector. Kappa light chain was ligated into PAX313m/hG vector digested with Xba I/BspE I. Transformation was performed in TOP10F′ cells (Invitrogen Life Technologies). Library size was 1.3×10⁸. Kappa light chain library in PAX313m/hG vector was used for the insertion of IgG1 and IgG2a fragments. Each fragment was ligated into kappa light chain library digested with Xho I/Bin 1. Transformation was performed in TOP10F′ cells. Titration of the library size was determined by plating the transformant on LB carbenicillin with glucose plates. The library sizes were 2.5×10⁹ for IgG1 kappa library and 1×10⁹ for IgG2a kappa library. The resultant colonies were used to see the fragment insertion and Fab expression by ELISA. Sixteen individual colonies from titration plates were inoculated into SB medium containing 50 μg/ml carbenicillin and grown for approximately 6 hrs at 37° C. A portion of the culture was diluted 1:100 in SB medium containing 50 μg/ml carbenicillin and 20 mM glucose and grown for overnight at 37° C. for Miniprep DNA purification. M13 VCS helper phage and 1 mM IPTG was added to the rest of the culture and grown for 2 hrs at 37° C. Kanamycin was added to the culture at 70 μg/ml and Fab-phage expression was induced overnight at 30° C. Fab expression ELISA was performed as described in EXAMPLE 4. The results of this experiment are shown in FIG 10. Fifteen out of sixteen IgG1 kappa library clones contained both heavy chain and kappa light chain inserts and 15 out of 16 IgG2a kappa library clones contained both heavy chain and light chain inserts. The library was panned on FLJ32028 peptide coated on microtiter wells using a method that is known for those skilled in the art (Maruyama T et al., J. Virol 73: 6024-6030, 1999). FIG. 11 shows the results of 4 rounds of panning. Fab expression was determined with wells coated with rabbit anti-mouse IgG F(ab′)₂ as described in EXAMPLE 4. The binding to the FLJ32028-Fc and mouse IgG Fc was also compared. Bound Fab was detected by alkaline phosphatase-conjugated goat anti-mouse IgG F(ab′)₂ antibody(1:500 in 1% BSAIPBS) (Pierce) as described in EXAMPLE 4. Most of the clones expressed very well (35 out of 48 clones showed OD405 above 3.0 in 30 minutes) and their binding to FLJ32028 antigen was very strong (20 out of 48 clones showed OD405 above 2.0 in 30 minutes) and specific (minimal binding to a control antigen mouse IgG Fc).

Although preferred and other embodiments of the invention have been described herein, further embodiments may be perceived by those skilled in the art without departing from the scope of the invention as defined by the following claims.

We claim:

1. An expression vector comprising nucleic acid encoding at least an antibody light chain region and an antibody heavy chain region and restriction sites for direct cloning of murine antibody genes into the vector, at least one of the antibody light chain region or the antibody heavy chain region being a hybrid regidn which is at least in part murine and at least in part human.

2. A hybrid light chain in an expression vector as in claim 1 wherein the antibody light chain constant region comprises at least a portion of a human kappa light chain constant region.

3. An expression vector as in claim 1 wherein the heavy chain constant region comprises at least a portion of a human heavy chain constant region CH1 domain.

4. An expression vector as in claim 1 wherein the heavy chain constant region comprises at least a portion of a human heavy chain constant region hinge domain.

5. An expression vector as in claim 1 wherein both the antibody light chain region and the antibody heavy chain region are hybrid regions which are at least in part murine and at least in part human.

6. A method of Fab expression comprising:
transfecting a host cell with an expression vector that comprising nucleic acid encoding at least an antibody light chain region and an antibody heavy chain region, at least one of the antibody light chain region or the antibody heavy chain region being a hybrid region which is at least in part murine and at least in part human,
wherein expression of Fabs is increased compared to the same vector having a purely murine constant regions.

7. An antibody comprising a hybrid constant region that is at least in part murine and at least in part human.

8. An antibody as in claim 7 wherein the hybrid constant region is the heavy chain constant region.

9. An antibody as in claim 7 wherein the hybrid constant region is the light chain constant region.

10. An antibody as in claim 7 wherein both the light chain constant region and the heavy chain constant region are hybrid constant regions.

11. An antibody as in claim 7 wherein the hybrid constant region includes at least a portion of a human heavy chain constant region CH1 domain.

12. An antibody as in claim 7 wherein the hybrid constant region includes at least a portion of a human heavy chain constant region hinge domain.

13. An antibody as in claim 7 wherein the hybrid constant region includes at least a portion of a human kappa light chain constant region.

14. A library of antibodies produced using an expression vector in accordance with claim 1.

15. A method of screening an antibody library comprising providing a library of antibodies in accordance with claim 14 and selecting antibodies having desired properties from said library.

16. In a method of expressing Fabs wherein a host cells in transfected with an expression vector containing nucleic acid encoding at least a portion of a murine heavy chain variable region and at least a portion of a murine heavy chain constant region, the improvement comprising substituting nucleic acid encoding at least a portion of a human heavy chain constant region for a portion of the nucleic acid encoding at least a portion of a murine heavy chain constant region, wherein Fabs expressed contain a hybrid heavy chain constant region.

17. In a method of expressing Fabs wherein a host cells in transfected with an expression vector containing nucleic acid encoding at least a portion of a murine light chain variable region and at least a portion of a murine light chain constant region, the improvement comprising substituting nucleic acid encoding at least a portion of a human light chain constant region for a portion of the nucleic acid encoding at least a portion of a murine light chain constant region, wherein Fabs expressed contain a hybrid light chain constant region.

18. In a method of expressing Fabs wherein a host cells in transfected with an expression vector containing nucleic acid encoding at least a portion of a murine light chain variable region and at least a portion of a murine light chain constant region, the improvement comprising substituting nucleic acid encoding at least a portion of a human light chain constant region for a portion of the nucleic acid encoding at least a portion of a murine light chain constant region and substituting nucleic acid encoding at least a portion of a human heavy chain constant region for a portion of the nucleic acid encoding at least a portion of a murine heavy chain constant region, wherein Fabs expressed contain a hybrid light chain constant region and a hybrid heavy chain constraint region.
